(19) United States
(12) Patent Application Publication
(10) Pub. No.: US 2007/0134287 A1
(43) Pub. Date: Jun. 14, 2007

(54) Method for coating biocompatible substrates with antibiotics

(75) Inventors: Karen Troxel, Warsaw, IN (US); Scott White, Leesburg, IN (US)

Correspondence Address: HARNESS, DICKEY & PIERCE, P.L.C., P.O. BOX 828, BLOOMFIELD HILLS, MI, 48303, US

(73) Assignee: Biomet Manufacturing Corp, Warsaw, IN (US)

(21) Appl. No.: 11/299,331

(22) Filed: Dec. 9, 2005

(51) Int. Cl. A61F 2/00; B05D 3/00

(52) U.S. Cl. 424/423; 427/2.24

(57) ABSTRACT

A method for coating a biocompatible substrate with an antibiotic is provided. The method includes dissolving at least one antibiotic in an antibiotic compatible solvent to provide an antibiotic solution. The antibiotic solution is applied to the substrate and the antibiotic compatible solvent is removed from the substrate. Methods of preparing a metallic implant and methods of providing localized antibiotic activity are also provided.

### FIELD

The present invention relates to methods for coating biocompatible substrates with antibiotics.

### BACKGROUND

Orthopedic devices and other prosthetic and implantable devices are potential targets for colonization of undesirable microorganisms. If an infection of the device occurs, the infection may be eradicated by removing the device and/or administering a course of antibiotics. After the infection is eradicated, a new joint prosthesis may be implanted.

To prevent infection, antimicrobial agents, such as antibiotics, may be bound to the surface of the device to produce sufficient bactericidal action to prevent colonization. Current technologies for coating antibiotics on the surface of implants require the use of a matrix (e.g., wax, silicone, or a film forming polymer) on the substrate to adhere the antibiotic to the implant. Using a matrix allows for incorporation of an antibiotic coating on a wide variety of implant substrate types including metal and polymer substrates having smooth surfaces or those with surface features. Other coating techniques are limited to only polymeric substrates such as where the antibiotic is incorporated into the polymer to provide a slow release of the antibiotic to the implant site.

Although these current techniques can be used with a variety of substrate types, the application method may interfere with bone or tissue ingrowth into certain devices. For example, with cementless bone implants, bone will not grow into the implant where polymeric surfaces are employed because these polymer coated surfaces do not serve as optimal binding sites for ingrowth of and strong bonding of new bone. Resorbable matrix coatings can similarly delay bone attachment to the metallic implant.

An additional concern with certain antibiotic coatings is displacement of the antibiotic coating during packaging operations. If the attachment of the antibiotics to the implant, with or without a binder, is weak, then the antibiotic coating may transfer to the packaging materials.

Accordingly, there is a need for methods of coating orthopedic devices and other prosthetic and implantable devices which do not interfere with bone or tissue ingrowth, provide localized antimicrobial activity, and are easily and safely packaged without compromising the antibiotic layer.

### SUMMARY

Methods for coating a metallic substrate with an antibiotic layer are provided. The methods include dissolving at least one antibiotic in an antibiotic compatible solvent to provide an antibiotic solution; applying the antibiotic solution to the metallic substrate; and removing the antibiotic compatible solvent from the metallic substrate. The methods may also include applying an additional antibiotic layer having a different antibiotic.

At least one antibiotic may be selected from macrolides and lincosamines, quinolones and fluoroquinolones, carbepenems, monobactams, aminoglycosides, glycopeptides, tetracyclines, sulfonamides, rifampins, oxazolidonones, and streptogramins, synthetic moieties thereof, and combinations thereof. The antibiotic compatible solvent may be selected from aqueous and organic solvents. The antibiotic solution may include at least one of minocycline and rifampin. The antibiotic compatible solvent may be methanol.

The application may be achieved by spraying, dipping, or spreading the antibiotic solution on at least a region of the metallic substrate. The antibiotic layer may have a density of less than about 800 μg/cm². The antibiotic layer may remain adhered to the metallic substrate by van der Waals bonding, ionic bonds, or hydrogen bonds between the antibiotic and the metallic substrate.

A method of preparing a metallic implant having an antibiotic layer is provided. The method comprises dissolving at least one antibiotic in an antibiotic compatible solvent to provide an antibiotic solution; applying the antibiotic solution to the metallic implant substrate; removing the antibiotic compatible solvent from the metallic implant substrate; and packaging the metallic implant without removing the antibiotic layer.

The antibiotic solution may include at least one of minocycline and rifampin. The packaging may include vacuum packaging the implant. The metallic implant may be adapted for adherence to bone and for bone ingrowth, may include a porous region, or may be adapted for cementless implantation into a subject.

Methods of providing localized antibiotic activity are also provided. The methods include providing a metallic implant having an antibiotic layer, where the antibiotic layer is applied directly to the metallic implant substrate; delivering the implant to an implant site; and hydrating the antibiotic layer to release the antibiotic to the implant site. The antibiotic layer may include at least one of minocycline and rifampin. The antibiotic layer may have a density of less than about 800 μg/cm². The hydrating may occur after delivering the implant to the implant site. The hydrating may be achieved by contacting the implant with fluids in the implant site.

Further areas of applicability of the present teachings will become apparent from the detailed description provided hereinafter. It should be understood that the detailed description and specific examples, while indicating the preferred embodiment of the teachings, are intended for purposes of illustration only and are not intended to limit the scope of the invention.

### BRIEF DESCRIPTION OF THE DRAWINGS

The present teachings will become more fully understood from the detailed description and the accompanying drawings, wherein:

FIG. 1 is a flow chart depicting a method of forming an implant;

FIG. 2 is a flow chart depicting a method of providing localized antibiotic activity; and

FIG. 3 depicts an implant according to various embodiments.

### DETAILED DESCRIPTION

The following description of the various embodiments is merely exemplary in nature and is in no way intended to limit the invention, its application, or uses.

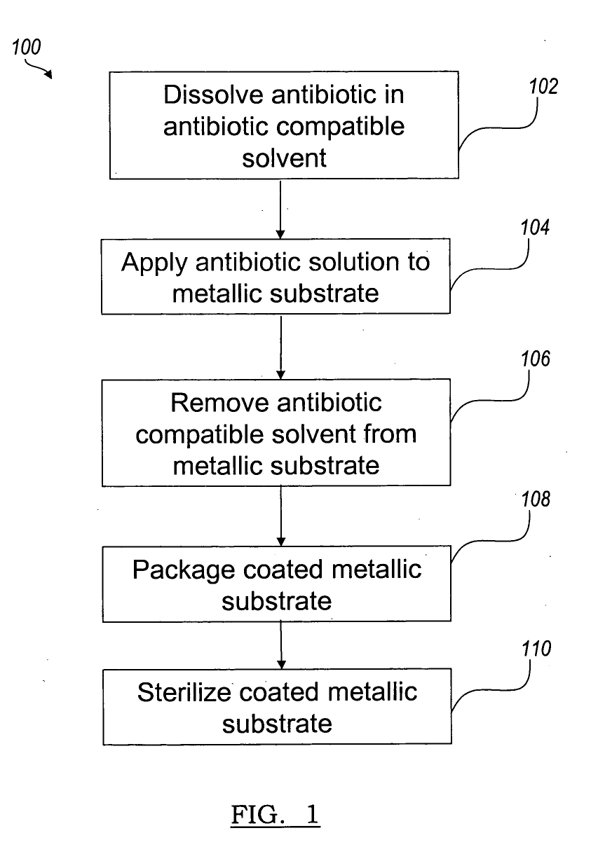

Referring to FIG. 1, various methods 100 of the present teachings provide an antibiotic coated metallic substrate. The methods include dissolving at least one antibiotic in an antibiotic compatible solvent to provide an antibiotic solution 102, applying the antibiotic solution to the metallic substrate 104, and removing the antibiotic compatible solvent from the metallic substrate 106. In various embodiments, the metallic substrate or metallic implant can be packaged 108 and sterilized 110. For simplicity, the methods relating to applying an antibiotic layer to a metallic substrate and the methods relating to providing a metallic implant having an antibiotic layer are discussed together.

Antibiotics (or antimicrobial) agents are effective in preventing the growth and eliminating the presence of bacterial and/or fungal organisms. The term “bacterial and fungal organisms” (or bacteria or fungi) as used herein refers to all genuses and species of bacteria and fungi, including but not limited to all spherical, rod-shaped, and spiral bacteria. Some examples of bacteria are stapylococci (i.e. Staphylococcus epidermidis, Staphylococcus aureus), Enterrococcus faecalis, Pseudomonas aeruginosa, Escherichia coli, other gram-positive bacteria and gram-negative bacilli. One example of a fungus is Candida albicans.

Antibiotics include the chemicals produced by one organism that are effective to inhibit the growth of another organism and include semi-synthetics, and synthetics thereof. As used herein, agents that reduce, inhibit, or prevent the growth or transmission of foreign organisms in a patient means that the growth or transmission of a foreign organism is reduced, inhibited, or prevented in a statistically significant manner in at least one clinical outcome, or by any measure routinely used by persons of ordinary skill in the art as a diagnostic criterion in determining the same.

Antibiotics may be selected from macrolides and lincosamines, quinolones and fluoroquinolones, carbepenems, monobactams, aminoglycosides, glycopeptides, tetracyclines, sulfonamides, rifampins, oxazolidonones, and streptogramins, synthetic moieties thereof, and combinations thereof. Example macrolides and lincosamines include azithromycin, clarithromycin, clindamycin, dirithromycin, erythromycin, lincomycin, and troleandomycin. Example quinolones and fluoroquinolones include cinoxacin, ciprofloxacin, enoxacin, gatifloxacin, grepafloxacin, levofloxacin, lomefloxacin, moxifloxacin, nalidixic acid, norfloxacin, ofloxacin, sparfloxacin, trovafloxacin, oxolinic acid, gemifloxacin, and perfloxacin. Example Carbepenes include imipenem-cilastatin and meropenem. Example monobactams include aztreonam. Example aminoglycosides include amikacin, gentamicin, kanamycin, neomycin, netilmicin, streptomycin, tobramycin, and paromomycin. Example glycopeptides include teicoplanin and vancomycin. Example tetracyclines include demeclocycline, doxycycline, methacycline, minocycline, oxytetracycline, tetracycline, and chlotetracycline. Example Sulfonamides include mafenide, silver sulfadizine, sulfacetamide, sulfadiazine, sulfamethoxazole, sulfasalazine, sulfisoxazole, trimethoprim-sulfamethoxazole, and sulfamethizole. An example oxazolidonone is linezolid. An example streptogramin is quinopristin+dalfopristin. Other suitable antibiotics include bacitracin, chloramphenicol, colistemetate, fosfomycin, isoniazid, methenamine, metronidazol, mupirocin, nitrofurantoin, nitrofurazone, novobiocin, polymyxin B, spectinomycin, trimethoprim, coliistin, cycloserine, capreomycin, ethionamide, pyrazinamide, para-aminosalicyclic acid, and erythromycin ethylsuccinate+sulfisoxazole. Still further antibiotics may also include the ample spectrum penicillins, penicillins and beta lactamase inhibitors, and cephalosporins. The antibiotics may be used alone or in combination.

In various embodiments, at least one of minocycline or rifampin is employed in the antibiotic solution. The application of the minocycline and rifampin combinations provide enhanced bactericidal activity of the metallic implant. Minocycline is primarily bacteriostatic and inhibits protein synthesis within a wide range of gram-positive and gram-negative organisms. Rifampin inhibits bacterial DNA-dependent RNA polymerase activity within a both gram-positive and gram-negative organisms.

The antibiotic compatible solvent may be selected from aqueous and organic solvents. As used herein, “antibiotic compatible” refers to the ability of the solvent to maintain the antibiotic in solution and the solution may be maintained without compromising the bactericidal activity of the antibiotic. The aqueous solvent may include sterile water. Other aqueous solvents include saline, balanced salt solutions, and phosphate buffered solutions, for example. Organic solvents include alcohols, ketones, ethers, aldehydes, acids, etc. Exemplary materials include TMSO, methylene chloride, chloroform, acetic acid, and the low molecular weight alcohols, such as methanol.

The selection of a particular antibiotic or antibiotics may determine the type of solvent employed. For example, minocycline may be provided as a hydrochloride salt which occurs as a yellow, crystalline powder. Minocycline is soluble in water and variously soluble in organic solvents. Rifampin, on the other hand, may be provided as a crystalline powder and is very slightly soluble in water and freely soluble in acidic aqueous solutions and organic solutions. One skilled in the art would appreciate that a combination of antibiotic layer comprising minocycline and rifampin would not be best soluble in water, due to the slight solubility of the rifampin in water and would employ an organic solvent, such as methanol, for example, to maximize solubility and application of the antibiotic solution on the metallic substrate.

The antibiotic may be in solution up to the solubility limit of the antibiotic and the solution. It may be advantageous to prepare a solution using an antibiotic and an antibiotic solvent in which the antibiotic is readily soluble. Providing a homogenous antibiotic solution through stirring or mixing may facilitate application and provide even coating of the antibiotic on the substrate, thereby increasing the antibacterial effectiveness of the substrate. In various embodiments, solution strength is chosen in a range close to the limit of solubility in order to provide maximum coating of the antibiotic while reducing the drying time or the time required to remove the solvent. Suitable antibiotic solution concentrations range from about 5 mg/mL up to about 30 mg/mL. For example, a 12 mg/mL solution of rampfin may provide an even coat of the antibiotic when applied using a spray applicator.

The antibiotic solution may be maintained a temperature conducive to retaining antibiotic activity for a specified amount of time during the application process. Generally, a desirable temperature range for maintaining or storing the antibiotic solution is from about −5° C. to about 40° C. The appropriate temperature range will depend on the cytotoxic effects of the antibiotic and the properties of the antibiotic compatible solvent. For example, it may be desirable to maintain an aqueous antibiotic solution comprising kanamycin sulfate at from about 2° C. to about 8° C. to maintain antibiotic stability. An antibiotic solution containing erythromycin in a 2M hydrochloric acid and alcohol solution may be optimized at about 25° C. The differences in temperature and solution type will affect the optimal conditions under which the antibiotic solution is applied. Referring to the kanamycin sulfate example, it may be desirable to apply the antibiotic solution in a cold room or to apply the antibiotic solution under time conditions which minimize warming the antibiotic solution until the process of removing the solvent.

The amount of antibiotic in the deposition layer is an amount sufficient to provide local antimicrobial activity after dissolution of the antibiotic into the tissues adjacent to the implant. The “amount sufficient to provide local antimicrobial activity” refers to the sufficient amount of the antibiotic to decrease, prevent or inhibit the growth of bacterial and/or fungal organism. The amount may vary for each antibiotic upon known factors such as pharmaceutical characteristics, the type of medical device, age, sex, health, and weight of the recipient, and the use and length of use of the coated substrate if implanted as a medical device.

Aqueous solutions of the antibiotic(s) may be prepared. Also, various water- and organic solvent-based solutions may contain pH buffering agents or salts. Selection of the antibiotic compatible solvent may include considerations of operator exposure.

The antibiotic solution may also include other therapeutic or healing agents to expedite healing, minimize infection or microbe colonization on the implant, or otherwise improve the function of the implant and/or the integration of the implant into the recipient. For example, the antibiotic solution may also include a protein such as a bone morphogenic protein.

Applying the antibiotic solution 104 may be achieved by spraying, dipping, or spreading the antibiotic solution on at least a region of the metallic substrate. As stated above, the antibiotic solution is applied in an amount sufficient to inhibit growth of bacteria and fungi. The antibiotic solution is maintained at a temperature sufficient to facilitate the particular application process. Suitable temperatures may include from about 10° C. up to about 75° C. The application of the antibiotic solution should generally be an even application to facilitate adherence of the antibiotic to the substrate and to prevent unintentional removal of the antibiotic layer. The thickness of the layer or coating density impacts the effectiveness of the methods. If the coating density is too thick, the antibiotic layer will come off onto the packaging. If the coating density is too thin, the level of bactericidal activity may be insufficient. Desired density of the antibiotic layer may be from about 1 μg/cm² to about 800 μg/cm². In various embodiments, the density is less than about 200 μg/cm², or less than about 100 μg/cm². In still other embodiments, the density is less than about 75 μg/cm². The antibiotic layer may be applied in a substantially uniform thickness.

The antibiotic layer may remain adhered to the metal by van der Waals bonding, or hydrogen bonds, ionic bonds, or other noncoralent bonds, between the antibiotic and the metal.

Suitable metallic substrates include any metal and may also include biocompatible metals. The metallic substrates may be selected from stainless steel, titanium, titanium alloys, tantalum, cobalt, cobalt alloys, and others. The metallic substrate may be prepared by traditional metal preparation techniques. For example, the substrate may be cleaned using standard cleaning protocol, particularly standard cleaning protocol employed with prosthetic implants. The metallic substrate may also be etched to provide an increased surface area. In embodiments where the metallic substrate has a porous surface or includes different surface features, it may be necessary to alter the standard application techniques (spraying a greater volume to coat pores, for example).

To remove the antibiotic compatible solvent 106, the metallic substrate coated with the antibiotic solution is dried to volatilize the solvent. The removal can be achieved by air drying to let the antibiotic compatible solvent leave in the air or the process can be expedited by using a drying oven. In embodiments where a drying oven is employed, it is desirable that the drying temperature be at a sufficiently low temperature to prevent denaturing or structural changes of the antibiotic. The drying may take a few second (from about 2 seconds to about 45 seconds), a few minutes (from about 2 minutes to about 45 minutes), or a few hours (from about 1 hour to about 5 hours). For example, in an embodiment where the antibiotic solution contains a very low concentration of the antibiotic dissolved in methanol, the drying time will generally be shorter than an aqueous solution saturated antibiotic solution.

The packaging 108 may include vacuum packaging the implant. The implant may be vacuum packaged into any suitable material, for example polyethylene, high density polyethylene, or nylon packaging. The process of vacuum packaging the implant and removing the implant from the vacuum packaging does not cause the antibiotic layer to become dislodged from or “flake off” of the implant. The sterilized implant is ready for use in the operating room. In various embodiments, the packaged implant may also be sterilized or heat treated 110 according to standard medical protocol. The metallic implant may be adapted for adherence to bone and for bone ingrowth, may include a porous region, or may be adapted for cementless implantation into a subject.

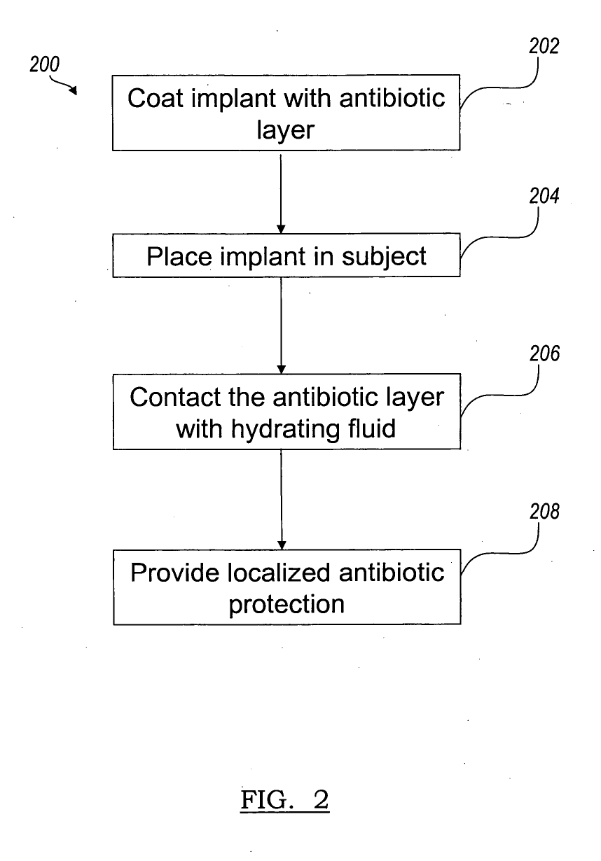

Referring to FIG. 2, methods of providing localized antibiotic activity 200 are also provided. The methods include providing a metallic implant having an antibiotic layer 202, where the antibiotic layer is applied directly to the metallic implant substrate without a film forming polymer or other matrix, delivering the implant to an implant site 204, and hydrating 206 the antibiotic layer to release the antibiotic to the implant site 208. The antibiotic layer may include at least one of minocycline and rifampin. The antibiotic layer may have a density of less than about 800 μg/cm². The thin antibiotic layer prevents the unintentional removal of the antibiotic from the metallic implant. In embodiments where the implant is packaged as disclosed above, the implant may be removed from the packaging without need for concern of unintentional transfer of the antibiotic layer to the packaging. The thin layer and the van der Waals forces between the antibiotic and the metallic substrate allow for the delivery of at least a substantial majority (greater than about 85%) of the antibiotic and provides enhanced localized antibiotic activity. Furthermore, without the presence of the polymer matrix or a wax coating to adhere to the antibiotic coating to the metallic substrate, the localized antibiotic activity may be provided with various implants which allow for the ingrowth of bone.

After making the necessary surgical incisions and preparing the implant area, the implant is inserted 204. Contact with the surrounding fluids in the implant site release the hydrogen bonds or van der Waals forces between the metallic substrate of the implant and the antibiotic layer to disperse the antibiotic to the surrounding tissue (localized delivery). Surrounding fluids include endogenous blood from the patient. The fluids may also be provided exogenously, such as by flushing the implant area containing the antibiotic coated implant with a saline solution or sterile water. The exogenous fluid may also include previously harvested blood from the patient or any blood product, including but not limited to platelet concentrate. The antibiotic is delivered to the implant site and is effective in preventing the colonization of bacteria at the implant surface in the implant site and near the point of incision. The antibiotic is delivered to the implant site in less than about 24 hours. In various embodiments, the antibiotic is delivered to the implant site in less than about 2 hours or less than about 2 minutes. The length of delivery time depends on the thickness of the layer and the solubility of the particular antibiotic. For example, a partly water-insoluble antibiotic will not dissolve from the implant into the surrounding tissue rapidly.

Using the antibiotic coated implant reduces several mechanisms of infection. By providing localized antibiotic activity at the time of implant (prosthetic, fixation plate, screws, or any implantable orthopedic device), the implant is protected from either direct or airborne contamination of the wound. The implant is also protected from an adjacent infection, such as that of the closed wound. Additionally, the implant is protected from any bacteremia or bacteria in the blood which may harbor at the implant site. Providing the localized antibiotic activity reduces, inhibits, and/or prevents the growth or transmission of foreign organisms in the patient. The even coating of the layer ensures that the antibiotic activity is dispersed throughout the implant region and is not limited to a single region of the implant.

In alternate embodiments, the delivery of the implant to provide local antibiotic effect may be combined with the delivery of a systemic antibiotic, such as an orally administered antibiotic. It may be desirable that the systemic antibiotic is synergistic with the localized antibiotic. This may provide the antibiotic effect that is desirable to prevent onset of infection in a new implant site. Additionally, an antibiotic ointment can be applied over the wound to prevent migration of bacteria through the wound and down towards the implant.

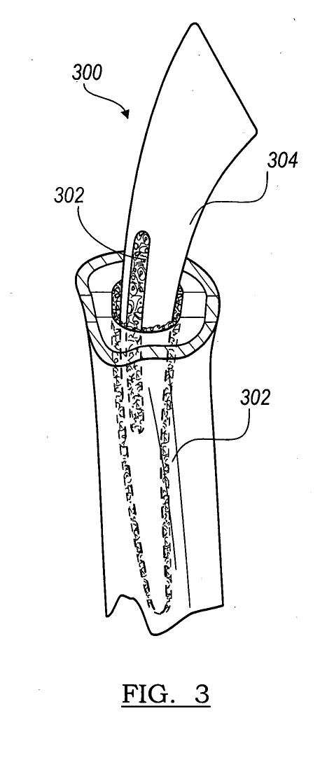

Referring to FIG. 3, in embodiments where the implant is a metallic implant used to repair bone defects without the use of cement, the antibiotic layer does not hinder the ingrowth of bone tissue into the implant. The femoral implant 300 includes porous regions 302 for the ingrowth of bone. The femoral implant 300 is coated with the ultra thin antibiotic layer 304 to prevent colonization of bacteria in the implant 300. While depicted as a cementless femoral stem, it is understood that medical device or implant may include hip, knee, elbow, shoulder, and wrist implants, fixation plates, screw, and the like. Other metallic devices may include non-orthopedic devices such as tracheostomy devices, intraurethanal and other genitourinary implants, stylets, dialators, stents, wire guides, and access ports of subcutaneously implanted vascular catheters.

The description of the invention is merely exemplary in nature and, thus, variations that do not depart from the gist of the invention are intended to be within the scope of the invention. Such variations are not to be regarded as a departure from the spirit and scope of the invention.

What is claimed is:

1. A method for coating a metallic substrate with an antibiotic layer, comprising:
a. dissolving at least one antibiotic in an antibiotic compatible solvent to provide an antibiotic solution;
b. applying the antibiotic solution to the metallic substrate; and
c. removing the antibiotic compatible solvent from the metallic substrate.

2. The method according to claim 1, wherein the at least one antibiotic is selected from the group consisting of: macrolides and lincosamines, quinolones and fluoroquinolones, carbepenems, monobactams, aminoglycosides, glycopeptides, tetracyclines, sulfonamides, rifampins, oxazolidonones, and streptogramins, synthetic moieties thereof, and combinations thereof.

3. The method according to claim 1, wherein the antibiotic compatible solvent is selected from aqueous solvents and organic solvents.

4. The method according to claim 1, wherein the applying is selected from spraying, dipping, or spreading the antibiotic solution on at least a region of the metallic substrate.

5. The method according to claim 1, wherein the antibiotic solution comprises at least one of minocycline and rifampin.

6. The method according to claim 5, wherein the antibiotic compatible solvent comprises methanol.

7. The method according to claim 1, wherein the antibiotic layer has a density of less than about 800 μg/cm².

8. The method according to claim 1, further comprising applying an additional antibiotic layer having a different antibiotic.

9. The method according to claim 1, wherein the antibiotic remains adhered to the metallic substrate by a bonding force selected from van der Waals forces, ionic bonding, or hydrogen bonding between the antibiotic and the metal.

10. A method of preparing a metallic implant having an antibiotic layer, comprising:
a. dissolving at least one antibiotic in an antibiotic compatible solvent to provide an antibiotic solution;
b. applying the antibiotic solution to the metallic implant substrate;
c. removing the antibiotic compatible solvent from the metallic implant substrate; and
d. packaging the metallic implant without removing the antibiotic layer.

11. The method according to claim 10, wherein the antibiotic solution comprises at least one of minocycline and rifampin.

12. The method according to claim 10, wherein said packaging comprises vacuum packaging the implant.

13. The method according to claim 10, wherein the metallic implant is adapted for adherence to bone and for bone ingrowth.

14. The method according to claim 10, wherein the metallic implant comprises a porous region.

15. The method according to claim 10, wherein the metallic implant is adapted for cementless implantation into a subject.

16. A method of providing localized antibiotic activity, comprising:
a. providing a metallic implant having an antibiotic layer, wherein the antibiotic layer is applied directly to the metallic implant substrate;
b. delivering the metallic implant to an implant site; and
c. hydrating the antibiotic layer to release the antibiotic to the implant site.

17. The method according to claim 16, wherein the antibiotic layer comprises at least one of minocycline and rifampin.

18. The method according to claim 16, wherein the antibiotic layer has a density of less than about 800 μg/cm².

19. The method according to claim 16, wherein the hydrating occurs after delivering the metallic implant to the implant site.

20. The method according to claim 16, wherein the hydrating comprises contacting the metallic implant with fluids in the implant site.
